(19) United States
(12) Patent Application Publication
(10) Pub. No.: US 2023/0255531 A1
(43) Pub. Date: Aug. 17, 2023

(54) STRESS COPING MANAGEMENT APPARATUS, STRESS COPING MANAGEMENT METHOD, AND COMPUTER-READABLE RECORDING MEDIUM

(71) Applicant: NEC Corporation, Minato-ku, Tokyo (JP)

(72) Inventor: Masanori TSUJIKAWA, Tokyo (JP)

(73) Assignee: NEC Corporation, Minato-ku, Tokyo (JP)

(21) Appl. No.: 18/012,167

(22) PCT Filed: Jun. 24, 2020
(86) PCT No.: PCT/JP2020/024791

§ 371 Date: Dec. 21, 2022

(51) Int. Cl. A61B 5/16; G16H 20/70; G16H 10/00

(52) U.S. Cl. CPC A61B 5/165; G16H 20/70; G16H 10/00

(57) ABSTRACT

The A stress coping management apparatus includes: a stress information obtaining unit that obtains stress information for specifying a stress level of a user; a stress coping candidate registration unit that registers a stress coping candidate in advance; a stress coping candidate selecting unit that selects a specific stress coping candidate from stress coping candidates based on the stress level of the user specified from the stress information; and a stress coping candidate presentation unit that presents the selected stress coping candidate. The stress coping candidate registration unit registers a new stress coping candidate input from a requester that has made the request to register the new stress coping candidate, and a stress level corresponding to the new stress coping candidate.

### TECHNICAL FIELD

The present invention relates to a stress coping management apparatus and a stress coping management method for presenting stress copings to a user, and furthermore relates to a computer-readable recording medium on which a program for realizing the apparatus and the method is recorded.

### BACKGROUND ART

In recent years, the working-age population has been decreasing due to the declining birthrate and the aging population, and the labor shortage has been becoming severer. In such a situation, an increasing number of attempts to replace a portion of tasks that have been manually performed to date with a robot or AI (Artificial Intelligence) have been made. Note that it is difficult to replace tasks that require intellectual labor among tasks that are performed by humans, with a robot or AI. Thus, also in the future, there is a need to maintain and improve the productivity of intellectual labor that is performed by humans.

Incidentally, unlike machines, humans are likely to be stressed, and, when humans are stressed, their capabilities decrease, and thus copings need to be taken for addressing the stress in order to maintain and improve the productivity of intellectual labor of humans. Thus, Patent Document 1 discloses an apparatus that evaluates the stress state of a human, and presents stress copings in accordance with the evaluation result.

Specifically, when a user inputs an answer to a questionnaire related to stress, the apparatus disclosed in Patent Document 1 presents copings such as “playing a sport”, “going to see a masseur”, “refraining from working on a holiday”, and “not staying up late”, as stress copings, based on past answers and past copings presented for the past answers.

### LIST OF RELATED ART DOCUMENTS

### Patent Document

Patent document 1: Japanese Patent No. 6426319

### SUMMARY OF INVENTION

### Problems to be Solved by the Invention

However, the above apparatus disclosed in Patent Document 1 can present only stress copings with a past proven record due to the configuration thereof, and thus such stress copings may be insufficient as stress copings depending on a user. A newly proposed or developed activity may be effective as stress copings for one user, for example. Also, an activity that no one has tried before may be effective as stress copings for another user.

An example object of the present invention is to provide a stress coping management apparatus, a stress coping management method, and a computer-readable recording medium that can solve the above problem, and present stress copings that are not restricted by past records, to a user.

### Means for Solving the Problems

In order to achieve the above-described object, a stress coping management apparatus includes:

a stress information obtaining unit that obtains stress information for specifying a stress level of a user;

a stress coping candidate registration unit registers a stress coping candidate in advance;

a stress coping candidate selecting unit that selects a specific stress coping candidate from stress coping candidates registered in advance, based on the stress level of the user specified from the stress information; and

a stress coping candidate presentation unit that presents the selected stress coping candidate,

wherein the stress coping candidate registration unit accepts a request to register a new stress coping candidate, and registers the new stress coping candidate input from a requester that has made the request to register the new stress coping candidate, and a stress level corresponding to the new stress coping candidate.

In addition, in order to achieve the above-described object, a stress coping management method includes:

a stress information obtaining step of obtaining stress information for specifying a stress level of a user;

a stress coping candidate registration step of registering a stress coping candidate in advance;

a stress coping candidate selecting step of selecting a specific stress coping candidate from stress coping candidates registered in advance, based on the stress level of the user specified from the stress information;

a stress coping candidate presentation step of presenting the selected stress coping candidate; and

wherein, in the stress coping candidate registration step, accepting a request to register a new stress coping candidate, and registering the new stress coping candidate input from a requester that has made the request to register the new stress coping candidate, and a stress level corresponding to the new stress coping candidate.

Furthermore, in order to achieve the above-described object, a computer readable recording medium according to an example aspect of the invention is a computer readable recording medium that includes recorded thereon a program,

the program including instructions that cause the computer to carry out:

a stress information obtaining step of obtaining stress information for specifying a stress level of a user;

a stress coping candidate registration step of registering a stress coping candidate in advance;

a stress coping candidate selecting step of selecting a specific stress coping candidate from stress coping candidates registered in advance, based on the stress level of the user specified from the stress information;

a stress coping candidate presentation step of presenting the selected stress coping candidate; and

wherein, in the stress coping candidate registration step, accepting a request to register a new stress coping candidate, and registering the new stress coping candidate input from a requester that has made the request to register the new stress coping candidate, and a stress level corresponding to the new stress coping candidate.

### Advantageous Effects of the Invention

As described above, according to the invention, it is possible to present stress copings that are not restricted by past records, to a user.

### BRIEF DESCRIPTION OF THE DRAWINGS

FIG. 1 is a block diagram showing the schematic configuration of the stress coping management apparatus according to the example embodiment.

FIG. 2 is a block diagram showing the configuration of the stress coping management apparatus according to the example embodiment in detail.

FIG. 3 is a flow diagram showing operations during processing for registering a stress coping candidate that is performed by the stress coping management apparatus according the example embodiment.

FIG. 4 is a flow diagram showing operations during the processing for presenting a stress coping candidate that is performed by the stress coping management apparatus according to the example embodiment.

FIG. 5 shows stress coping candidates registered in advance and selected stress coping candidates in Specific Example 1.

FIG. 6 shows stress coping candidates registered in advance and selected stress coping candidates in Specific Example 2.

FIG. 7 shows stress coping candidates registered in advance and selected stress coping candidates in Specific Example 3.

FIG. 8 shows stress coping candidates registered in advance and selected stress coping candidates in Specific Example 4.

FIG. 9 is a block diagram illustrating an example of a computer that realizes the stress coping management apparatus according to the example embodiment.

### EXAMPLE EMBODIMENT

### Example Embodiment

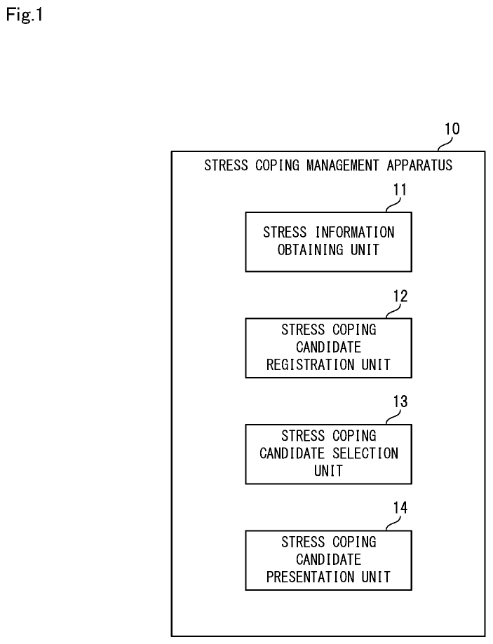

A stress coping management apparatus, a stress coping management method, and a program according to an example embodiment of the present invention will be described below with reference to FIGS. 1 to 9.

### Apparatus Configuration

First, a schematic configuration of the stress coping management apparatus according to the example embodiment will be described with reference to FIG. 1. FIG. 1 is a block diagram showing the schematic configuration of the stress coping management apparatus according to the example embodiment.

The stress coping management apparatus 10 according to the example embodiment shown in FIG. 1 is an apparatus for presenting stress copings in accordance with the stress level of the user. As shown in FIG. 1, the stress coping management apparatus 10 includes a stress information obtaining unit 11, a stress coping candidate registration unit 12, a stress coping candidate selection unit 13, and a stress coping candidate presentation unit 14.

The stress information obtaining unit 11 obtains stress information for specifying the stress level of the user. The stress coping candidate registration unit 12 registers stress coping candidates in advance. The stress coping candidate selection unit 13 selects a specific stress coping candidate from the stress coping candidates registered in advance, based on the stress level of the user specified based on the stress information. The stress coping candidate presentation unit 14 presents the selected stress coping candidate.

Moreover, the stress coping candidate registration unit 12 accepts a request to register a new stress coping candidate, and registers the new stress coping candidate input by the requester who has requested that the new stress coping candidate be registered, and a stress level corresponding to the new stress coping candidate.

In this manner, in the example embodiment, a stress coping candidate corresponding to the stress level of the user is presented to the user. Moreover, in the example embodiment, a new stress candidate can also be registered. Therefore, according to the example embodiment, it is possible to present stress copings that are not restricted by past records, to the user.

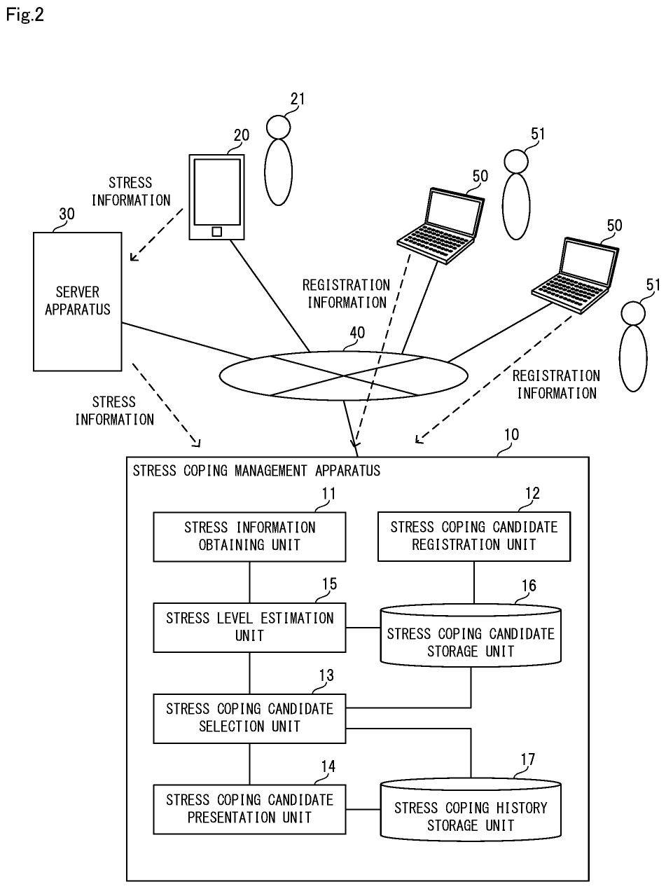

Next, the configuration and functions of the stress coping management apparatus according to the example embodiment will be described in detail with reference to FIG. 2. FIG. 2 is a block diagram showing the configuration of the stress coping management apparatus according to the example embodiment in detail.

As shown in FIG. 2, in the example embodiment, the stress coping management apparatus 10 includes a stress level estimation unit 15, a stress coping candidate storage unit 16, and a stress coping history storage unit 17, in addition to the stress information obtaining unit 11, the stress coping candidate registration unit 12, the stress coping candidate selection unit 13, and the stress coping candidate presentation unit 14, which have been described above.

In addition, as shown in FIG. 2, in the example embodiment, the stress coping management apparatus 10 is connected to a server apparatus 30 via a network 40 such as the Internet. The server apparatus 30 is a Web server that provides a Web site serving as an interface between the stress coping management apparatus 10 and a user 21. The user 21 accesses the server apparatus 30 from a terminal apparatus 20 of the user 21, and inputs, on the Web site, stress information for specifying a stress level. Accordingly, the server apparatus 30 transmits the input stress information to the stress coping management apparatus 10. Note that physically the same apparatus may have the functions of the server apparatus 30 and the stress coping management apparatus 10.

In the example embodiment, the stress information obtaining unit 11 obtains stress information transmitted from the server apparatus 30. The stress information may be any information that enables the stress level to be specified, and is not particularly limited. Examples of stress information include biological information. The stress information may also be a numerical value indicating a stress level.

If the stress information includes biological information, the stress level estimation unit 15 estimates a stress level based on the biological information. Specifically, the biological information may be a heart rate, electrodermal activity, the amount of perspiration, a face image, an acceleration rate indicating movement of a human, or the like. The biological information may be obtained based on information from a wearable sensor or a sensor such as a camera.

The stress level estimation unit 15 estimates a stress level using an existing technique and a technique to be developed in the future depending on the type of biological information, for example. Note that a technique for estimating a stress level if the biological information is an acceleration rate indicating movement of the body of the user, an electrodermal activity indicating a perspiration state, or a cutaneous temperature may be the technique disclosed in Reference Document 1 or 2 to be described later. In addition, biological information that is used by the stress level estimation unit 15 is not limited to the above, and various types of biological information are used.

Reference Document 1: A. Sano et al., “Recognizing academic performance, sleep quality, stress level, and mental health using personality traits, wearable sensors and mobile phones,” in Wearable and Implantable Body Sensor Networks (BSN), 2015 IEEE 12th International Conference on, 2015, pp. 1-6.

Reference Document 2: Yoshiki Nakashima and two others, “Improvement in Chronic Stress Level Recognition by Using Both Full-term and Short-term Measurements of Physiological Features”, The 32nd Annual Conference of the Japanese Society for Artificial Intelligence, 2018.

In the example embodiment, a vendor (or an individual) 51 that provides stress coping candidates requests that a new stress coping candidate be registered, from a terminal apparatus 50 thereof via the network 40. In this case, the stress coping candidate registration unit 12 obtains information regarding the new stress coping candidate input by the requester that has requested that the stress coping candidate be registered. The stress coping candidate registration unit 12 then registers the stress coping candidate by storing the obtained information regarding the new stress coping candidate to the stress coping candidate storage unit 16.

Information regarding a stress coping candidate is information for specifying the content of the stress coping candidate. In addition, in the example embodiment, the vendor 51 can set a stress level corresponding to a stress coping candidate for which the vendor 51 is to make a request for registration, as additional information, and request that the stress coping candidate be registered. In this case, information regarding the stress coping candidate that is stored in the stress coping candidate storage unit 16 includes information regarding the corresponding stress level.

Examples of a stress coping candidate include hospitals, counseling, massage, gyms, hot springs, travel, stress relief items, and the like. Note that, in the example embodiment, any stress coping candidate that can relieve stress of a human can be used, and the degree of relief does not matter.

Furthermore, assume that, after the stress coping candidate selection unit 13 selects a stress coping candidate, and the stress coping candidate presentation unit 14 presents the stress coping candidate, stress information of the user to which the stress coping candidate was presented is obtained again. In this case, the stress coping candidate registration unit 12 can notify the requester (the vendor 51) that had made a request to register the selected stress coping candidate, of stress levels of the user before and after the stress coping candidate was selected.

In the example embodiment, the stress coping candidate selection unit 13 specifies stress levels corresponding to stress coping candidates stored in the stress coping candidate storage unit 16, based on information regarding the stress coping candidates. The stress coping candidate selection unit 13 then selects a stress coping candidate for which the corresponding stress level matches the stress level of the user, from the specified stress coping candidates.

In the example embodiment, the stress coping candidate presentation unit 14 presents the stress coping candidate selected by the stress coping candidate selection unit 13, to the terminal apparatus 20 of the user 21. Specifically, the stress coping candidate presentation unit 14 transmits the selected stress coping candidate to the server apparatus 30, and presents the selected stress coping candidate on the screen of the terminal apparatus 20 of the user 21, using the Web site provided by the server apparatus 30.

In addition, in the example embodiment, when there are a plurality of stress coping candidates that are to be presented, the above-described stress coping candidate selection unit 13 can calculate a matching degree between a stress level corresponding to each of the stress coping candidates and the stress level of the user. Furthermore, in calculation of a matching degree, the stress coping candidate selection unit 13 can also take information other than a stress level into consideration. If, for example, information regarding a stress coping candidate registered in the stress coping candidate storage unit 16 includes a gender, an age, and the like of the corresponding user, the stress coping candidate selection unit 13 can calculate a matching degree in consideration of the user's gender, age, and the like.

Moreover, if matching degrees are calculated in this manner, the stress coping candidate presentation unit 14 can present a plurality of stress coping candidates such that a stress coping candidate with a higher matching degree is more highly prioritized.

The stress coping history storage unit 17 stores, as a stress coping history, the stress level of the user, stress coping candidates presented to the user, and a stress coping candidate in which the user shows interest, in a state of being associated with one another. Note that whether or not the user shows interest is determined using an existing technique such as a technique for detecting whether or not the user has clicked on a presented stress coping candidate.

### Apparatus Operations

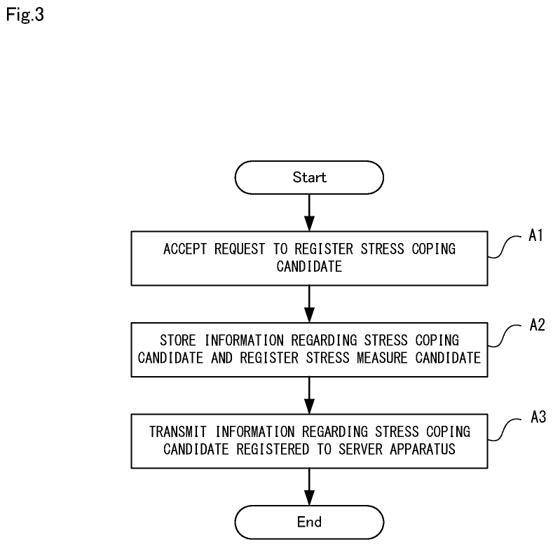

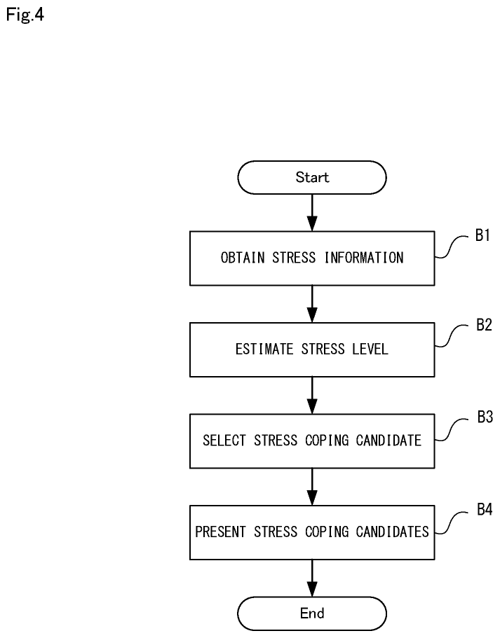

Next, operations of the stress coping management apparatus according to the example embodiment will be described with reference to FIGS. 3 and 4. In the following description, FIGS. 1 and 2 will be referred to as appropriate. In addition, in a first example embodiment, a stress coping management method is carried out by causing the stress coping management apparatus 10 to operate. Thus, a description of the stress coping management method according to the example embodiment is replaced by the following description of operations of the stress coping management apparatus 10.

First, processing for registering a stress coping candidate will be described with reference to FIG. 3. FIG. 3 is a flow diagram showing operations during processing for registering a stress coping candidate that is performed by the stress coping management apparatus according the example embodiment.

As shown in FIG. 3, first, if the vendor 51 that provides stress coping candidates requests that a stress coping candidate be registered, from the terminal apparatus 50 thereof via the network 40, the stress coping candidate registration unit 12 accepts the request to register the stress coping candidate (step A1). In step A1, a certain condition may be set for accepting a request to register a stress coping candidate.

Next, the stress coping candidate registration unit 12 stores information regarding the requested stress coping candidate, to the stress coping candidate storage unit 16, thereby registering the stress coping candidate (step A2). Note that the information regarding the stress coping candidate also includes information regarding the corresponding stress level.

Next, the stress coping candidate registration unit 12 transmits the information regarding the stress coping candidate registered in step A2, to the server apparatus 30 (step A3). Accordingly, the newly registered stress coping candidate and the corresponding stress level are displayed on the screen of the terminal apparatus 20 of the user 21.

Next, processing for presenting a stress coping candidate will be described with reference to FIG. 4. FIG. 4 is a flow diagram showing operations during the processing for presenting a stress coping candidate that is performed by the stress coping management apparatus according to the example embodiment.

First, if the user 21 accesses the server apparatus 30 from the terminal apparatus 20 thereof, and inputs stress information, the server apparatus 30 transmits the stress information to the stress coping management apparatus 10. Accordingly, as shown in FIG. 4, in the example embodiment, the stress information obtaining unit 11 obtains the stress information transmitted from the server apparatus 30 (step B1).

Next, since the stress information includes biological information, the stress level estimation unit 15 estimate a stress level based on the biological information included in the biological information (step B2).

Next, the stress coping candidate selection unit 13 specifies stress levels corresponding to stress coping candidates stored in the stress coping candidate storage unit 16, based on the information regarding the stress coping candidates. The stress coping candidate selection unit 13 then selects a stress coping candidate for which the corresponding stress level matches the stress level of the user estimated in step B2, from the specified stress coping candidates (step B3).

Moreover, in step B3, when there are a plurality of stress coping candidates to be presented, the stress coping candidate selection unit 13 can calculate a matching degree between a stress level corresponding to each of the stress coping candidates and the stress level of the user. Furthermore, in calculation of a matching degree, the stress coping candidate selection unit 13 can also take information other than a stress level into consideration.

Next, the stress coping candidate presentation unit 14 presents the stress coping candidates selected in step B3, on the terminal apparatus 20 of the user 21 (step B4). Specifically, the stress coping candidate presentation unit 14 transmits the selected stress coping candidates to the server apparatus 30, and presents the selected stress coping candidates on the screen of the terminal apparatus 20 of the user 21 using the Web site provided by the server apparatus 30.

In addition, if the stress coping candidate selection unit 13 calculates a matching degree for each of the stress coping candidates in step B3, the stress coping candidate presentation unit 14 presents, in step B4, the plurality of stress coping candidates such that a stress coping candidate with a higher matching degree is more highly prioritized.

After step B4 is executed, if the user selects a stress coping candidate suitable to the user and actually executes the stress coping candidate, the user 21 can input information regarding the used stress coping candidate to the server apparatus 30 via the Web site.

In addition, the stress coping candidate selection unit 13 stores, as a stress coping history, the stress level of the user, the stress coping candidates presented to the user, and a stress coping candidate in which the user showed interest, in a state of being associate with one another, to the stress coping history storage unit 17. Note that the stress coping candidate selection unit 13 can determine whether or not the user has shown interest, using an existing technique for detecting whether or not the user has clicked on a presented stress coping candidate.

### MODIFIED EXAMPLES

Here, Modified Examples 1 to 4 of the example embodiment will be described.

### Modified Example 1

In Modified Example 1, in registration of a stress coping candidate, the vendor 51 can designate a user state in which the stress coping candidate is effective. The user state that is designated may be a state where the stress level is increasing, a state where the stress level is maintained, a state where the stress level is decreasing, or the like. In Modified Example 1, the stress coping candidate storage unit 16 stores each stress coping candidate, a corresponding stress level (presentation target), and a corresponding user state.

In this case, in selection of a stress coping candidate, the stress coping candidate selection unit 13 obtains a past stress level of the target user from the stress coping history storage unit 17, and compares the obtained past stress level with the most recent stress level, and specifies a user state. The stress coping candidate selection unit 13 selects a stress coping candidate from the stress coping candidates stored in the stress coping candidate storage unit 16, based on the most recent stress level and the user state.

### Modified Example 2

In Modified Example 2, the server apparatus 30 obtains position information for specifying the position of the user (the position of the terminal apparatus 20), from the user 21 via the terminal apparatus 20, and transmits the obtained position information along with stress information to the stress coping management apparatus 10.

In Modified Example 2, the stress information obtaining unit 11 obtains position information for specifying the position of the user in addition to stress information. In addition, the stress coping candidate registration unit 12 registers a stress coping candidate along with information regarding an area in which the stress coping candidate can be used. Furthermore, the stress coping candidate selection unit 13 specifies stress coping candidates that can be carried out, based on the position of the user specified based on the position information, and areas in which the stress coping candidates can be used. The stress coping candidate selection unit 13 then selects a specific stress coping candidate from the specified stress coping candidates, based on the stress level of the user.

According to Modified Example 2, an appropriate stress coping candidate can be presented to the user in accordance with the location of the user.

### Modified Example 3

In Modified Example 3, the server apparatus 30 obtains time information regarding a time (a.m., p.m., evening, a day of the week, etc.) when the user desires stress copings, from the user 21 via the terminal apparatus 20, and transmits the obtained time information along with stress information to the stress coping management apparatus 10.

In Modified Example 3, the stress information obtaining unit 11 obtains time information regarding a time when the user desires stress copings, in addition to stress information. Moreover, the stress coping candidate registration unit 12 registers a stress coping candidate along with information regarding a time when the stress coping candidate can be used. Furthermore, the stress coping candidate selection unit 13 specifies stress coping candidates that can be carried out, based on the time when the user desires stress copings, the time being specified based on the time information, and times when stress coping candidates can be used. The stress coping candidate selection unit 13 then selects a specific stress coping candidate from the specified stress coping candidates, based on the stress level of the user.

According to Modified Example 3, appropriate stress coping candidates can be presented to the user in accordance with the schedule of the user.

### Modified Example 4

In Modified Example 4, when the user 21 actually uses a stress coping candidate, the server apparatus 30 obtains information regarding the stress coping candidate used by the user, from the user 21 via the terminal apparatus 20, and transmits the obtained information along with stress information to the stress coping management apparatus 10. Examples of information regarding a stress coping candidate used by the user include a name (or an identifier) thereof, use time and date, evaluation (effective or not effective), and the like.

In Modified Example 4, the stress information obtaining unit 11 obtains the information regarding the stress coping candidate used by the user 21, and stores the obtained information to the stress coping history storage unit 17. In this case, the stress coping candidate registration unit 12 can set a weight (recommended level) for each stress coping candidate, based on the information regarding the stress coping candidate used by the user 21. Note that the initial value of a weight may be set by the administrator of the stress coping management apparatus 10 as appropriate. In addition, the stress coping candidate registration unit 12 can set a weight (recommendation level) using information regarding a stress coping candidate used by a user other than the user 21.

Moreover, if a plurality of stress coping candidates have been selected, and a weight is set as described above, the stress coping candidate presentation unit 14 can set a priority of each of the selected stress coping candidates in accordance with the weight. In this case, the stress coping candidate presentation unit 14 presents the selected stress coping candidates in priority order, on the screen of the terminal apparatus 20

Furthermore, in Modified Example 4, the stress coping candidate registration unit 12 can also specify the number of times a presented stress coping candidate was used by the user, based on information regarding the stress coping candidate used by the user 21. In this case, the stress coping candidate registration unit 12 sets a cost of registration of the stress coping candidate, based on the specified number of times. As a result, for example, the larger the number of times is, the more money the vendor 51 earns, and thus a higher cost of registration the vendor 51 pays.

### Specific Examples

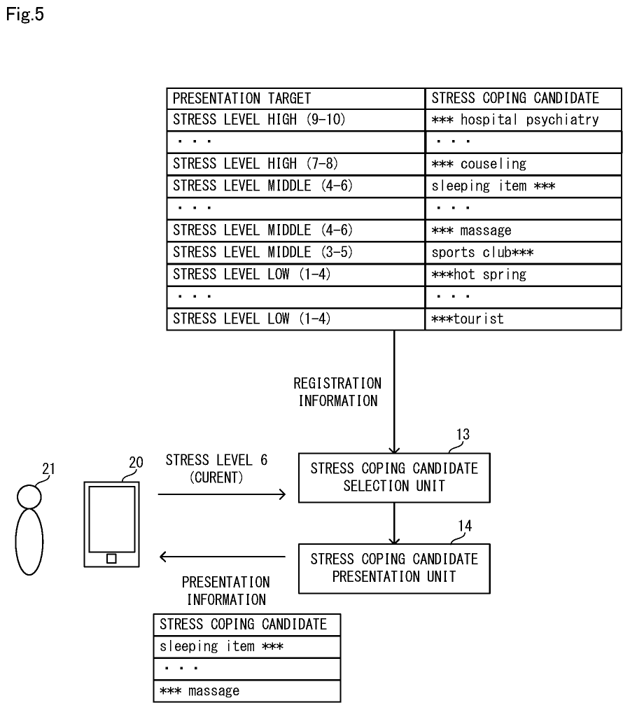

Next, Specific Examples 1 to 4 will be described with reference to FIGS. 5 to 8. FIGS. 5 to 8 show stress coping candidates registered in advance and selected stress coping candidates in Specific Examples.

### Specific Example 1

As shown in FIG. 5, in Specific Example 1, the stress coping candidate storage unit 16 stores, as information, stress coping candidates and corresponding stress levels (presentation targets). The most recent stress level of the user is 6.

Therefore, the stress coping candidate selection unit 13 selects “sleeping item ***” and “*** massage” as stress coping candidates, and the stress coping candidate presentation unit 14 displays the selected stress coping candidates on the screen of the terminal apparatus 20 via the server apparatus 30.

### Specific Examples 2

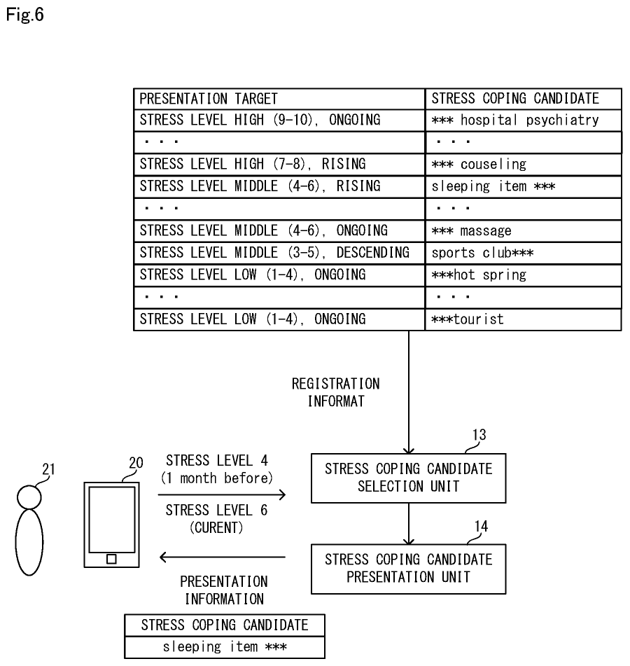

As shown in FIG. 6, in Specific Example 2, the stress coping candidate storage unit 16 stores, as information, stress coping candidates and corresponding stress levels and user states (presentation targets) (see Modified Example 1). While the most recent stress level of the user is 6, the stress level obtained one month before and stored in the stress coping history storage unit 17 is 4, which means the stress level is increasing.

Therefore, the stress coping candidate selection unit 13 selects only “sleeping item ***” as a stress coping candidate, and the stress coping candidate presentation unit 14 displays the selected stress coping candidate on the screen of the terminal apparatus 20 via the server apparatus 30.

### Specific Example 3

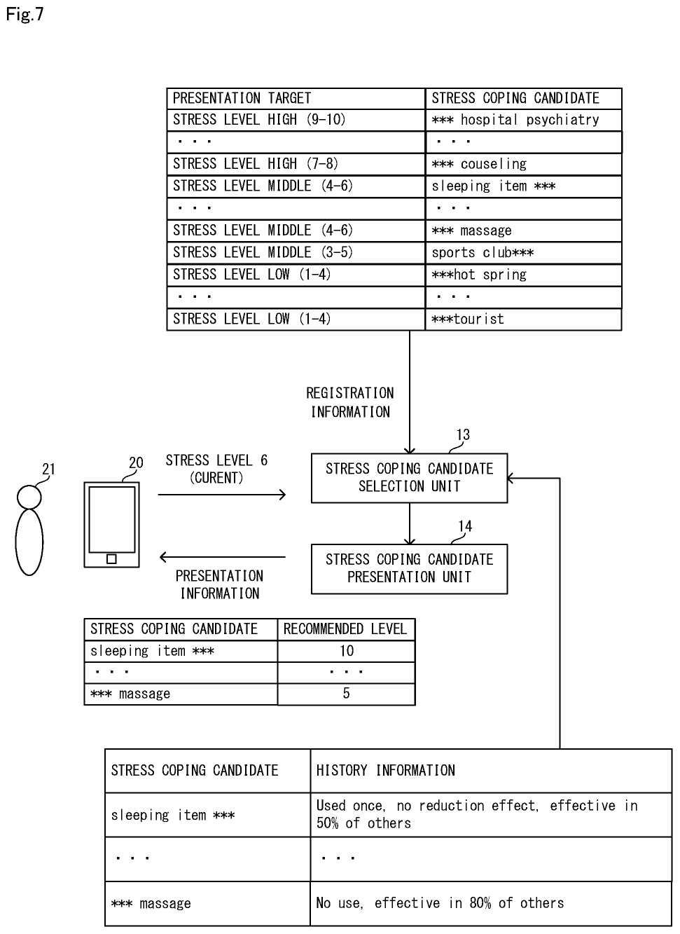

In Specific Example 3 shown in FIG. 7, similarly to Specific Example 1, the stress coping candidate storage unit 16 stores, as information, stress coping candidates and corresponding stress levels (presentation targets). The most recent stress level of the user is 6.

Note that, unlike Specific Example 1, the stress coping history storage unit 17 stores information regarding stress coping candidates used by the user 21, as history information (see Modified Example 4). In addition, the stress coping candidate registration unit 12 sets a weight (recommendation level) for each stress coping candidate, based on the information regarding the stress coping candidate used by the user 21 (see Modified Example 4).

Therefore, similarly to Specific Example 1, the stress coping candidate selection unit 13 selects “sleeping item ***” and “*** massage” as stress coping candidates, while the stress coping candidate presentation unit 14 sets a priority of each of the selected stress coping candidates based on the weight, and displays the stress coping candidates on the screen of the terminal apparatus 20 in priority order.

### Specific Example 4

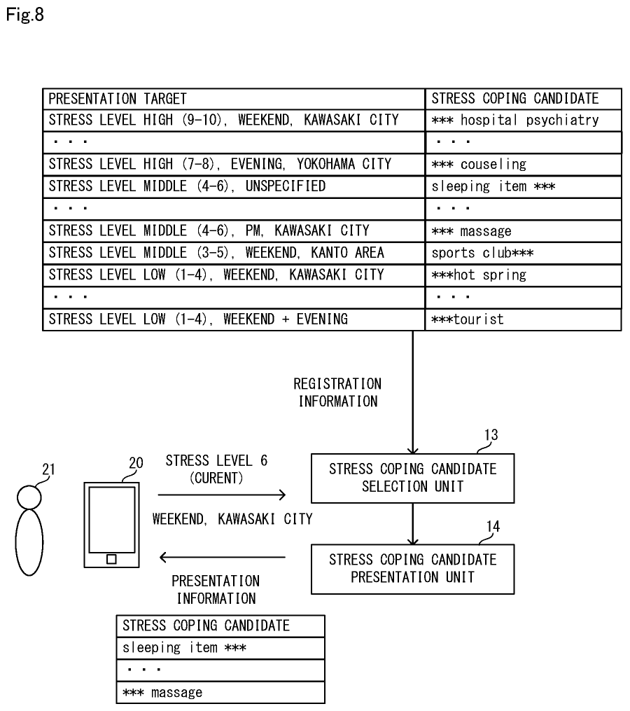

As shown in FIG. 8, in Specific Example 4, in addition to stress coping candidates and corresponding stress levels (presentation targets), the stress coping candidate storage unit 16 also stores position information of the user and time information regarding a time when the user desires stress copings, as information (see Modified Examples 2 and 3). The most recent stress level of the user is 6, and furthermore, the user is positioned in Kawasaki-city. In addition, a user's desired time is 16:00.

Therefore, the stress coping candidate selection unit 13 selects “sleeping item ***” and “*** massage” as position stress coping candidates based on the stress level of the user, the position of the user, and the time when the user desires stress copings. The stress coping candidate presentation unit 14 displays the selected stress coping candidates on the screen of the terminal apparatus 20 via the server apparatus 30.

### Effects According to Example Embodiment

As described above, according to the example embodiment, it is possible to select a stress coping candidate for the user in accordance with the stress level of the user, and to present this stress coping candidate to the user, and thus the user can appropriately manage their stress.

Moreover, in the example embodiment, a past stress level and a stress coping candidate corresponding to the past stress level are accumulated as history, and thus it is also possible to present a stress coping candidate suitable to the user using past information as well.

Furthermore, in the example embodiment, the vendor 51 can register their business as a stress coping candidate in accordance with a stress level, and thus it is possible to present various stress coping candidates to the user.

In addition, in the example embodiment, it is also possible to specify a change in the stress level of the user, and thus it is also possible to present an effective stress coping candidate in accordance with the state of the user. Furthermore, in the example embodiment, it is also possible for the vendor 51 that provides stress coping candidates to correct registration information thereof, and a stress coping candidate that can be provided, afterward.

### Program

It suffices for a program in the example embodiment to be a program that causes a computer to carry out steps A1 to A3 illustrated in FIG. 3 and steps B1 to B4 illustrated in FIG. 4. Also, by this program being installed and executed in the computer, the stress coping management apparatus and the stress coping management method according to the example embodiment can be realized. In this case, a processor of the computer functions and performs processing as the stress information obtaining unit 11, the stress coping candidate registration unit 12, the stress coping candidate selection unit 13, the stress coping candidate presentation unit 14, and the stress level estimation unit 15.

In the example embodiment, the stress coping candidate storage unit 16 and the stress coping history storage unit 17 may be realized by storing the data files constituting this in a storage device such as a hard disk provided in the computer. Also, the stress coping candidate storage unit 16 and the stress coping history storage unit 17 may be realized by a storage device of another computer. The computer that constructs the stress coping management apparatus includes general-purpose PC, smartphone and tablet-type terminal device. The computer that constructs the stress coping management apparatus may be the server apparatus 30 illustrated in FIG. 2.

Furthermore, the program according to the example embodiment may be executed by a computer system constructed with a plurality of computers. In this case, for example, each computer may function as one of the stress information obtaining unit 11, the stress coping candidate registration unit 12, the stress coping candidate selection unit 13, the stress coping candidate presentation unit 14, and the stress level estimation unit 15.

### Physical Configuration

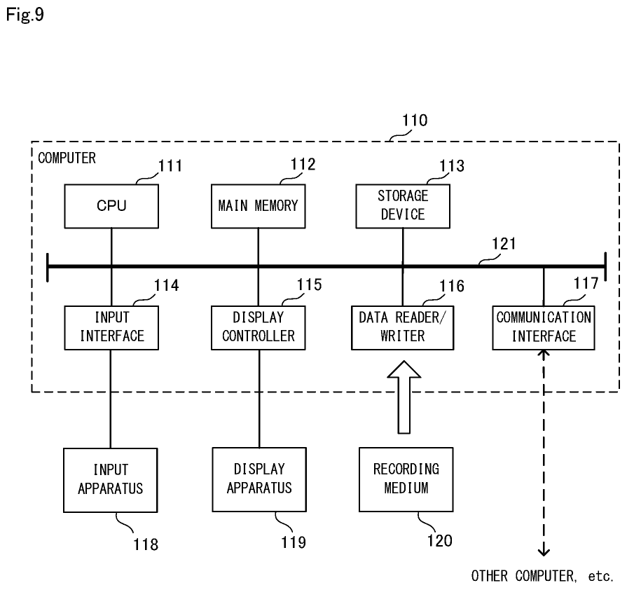

Using FIG. 9, the following describes a computer that realizes the stress coping management apparatus 10 by executing the program according to the example embodiment. FIG. 9 is a block diagram illustrating an example of a computer that realizes the stress coping management apparatus according to the example embodiment.

As shown in FIG. 9, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121.

The computer 110 may include a GPU (Graphics Processing Unit) or an FPGA (Field-Programmable Gate Array) in addition to the CPU 111, or in place of the CPU 111. In this case, the GPU or the FPGA can execute the programs according to the example embodiment.

The CPU 111 deploys the program according to the example embodiment, which is composed of a code group stored in the storage device 113 to the main memory 112, and carries out various types of calculation by executing the codes in a predetermined order. The main memory 112 is typically a volatile storage device, such as a DRAM (dynamic random-access memory).

Also, the program according to the example embodiment is provided in a state where it is stored in a computer-readable recording medium 120. Note that the program according to the present example embodiment may be distributed over the Internet connected via the communication interface 117.

Also, specific examples of the storage device 113 include a hard disk drive and a semiconductor storage device, such as a flash memory. The input interface 114 mediates data transmission between the CPU 111 and an input device 118, such as a keyboard and a mouse. The display controller 115 is connected to a display device 119, and controls display on the display device 119.

The data reader/writer 116 mediates data transmission between the CPU 111 and the recording medium 120, reads out the program from the recording medium 120, and writes the result of processing in the computer 110 to the recording medium 120. The communication interface 117 mediates data transmission between the CPU 111 and another computer.

Specific examples of the recording medium 120 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the stress coping management apparatus 10 according to the example embodiment can also be realized by using items of hardware that respectively correspond to the components, rather than the computer in which the program is installed. Furthermore, a part of the stress coping management apparatus 10 may be realized by the program, and the remaining part of the stress coping management apparatus 10 may be realized by hardware.

A part or an entirety of the above-described example embodiment can be represented by (Supplementary Note 1) to (Supplementary Note 24) described below, but is not limited to the description below.

### Supplementary Note 1

A stress coping management apparatus including:

a stress information obtaining unit that obtains stress information for specifying a stress level of a user;

a stress coping candidate registration unit that registers a stress coping candidate in advance;

a stress coping candidate selecting unit that selects a specific stress coping candidate from stress coping candidates registered in advance, based on the stress level of the user specified from the stress information; and

a stress coping candidate presentation unit that presents the selected stress coping candidate,

wherein the stress coping candidate registration unit accepts a request to register a new stress coping candidate, and registers the new stress coping candidate input from a requester that has made the request to register the new stress coping candidate, and a stress level corresponding to the new stress coping candidate.

### Supplementary Note 2

The stress coping management apparatus according to Supplementary Note 1,

wherein the stress coping candidate registration unit sets a weight for each stress coping candidate, and

when a plurality of stress coping candidates are selected, the stress coping candidate presentation unit sets a priority of each of the plurality of selected stress coping candidates in accordance with the weight, and presents the selected stress coping candidates in priority order.

### Supplementary Note 3

The stress coping management apparatus according to Supplementary Note 1 or 2,

wherein the stress coping candidate registration unit notifies a requester that has made a request to register the selected stress coping candidate, of stress levels of the user before and after the stress coping candidate is selected.

### Supplementary Note 4

The stress coping management apparatus according to any one of Supplementary Notes 1 to 3, further comprising:

a stress level estimation unit that estimates a stress level based on biological information of the user if the stress information includes the biological information,

wherein the stress coping candidate selecting unit selects a specific stress coping candidate based on the stress level estimated from the biological information.

### Supplementary Note 5

The stress coping management apparatus according to any one of Supplementary Notes 1 to 4,

wherein the stress information obtaining unit further obtains position information for specifying a position of the user,

the stress coping candidate registration unit registers a stress coping candidate along with information regarding an area where the stress coping candidate can be used, and

the stress coping candidate selecting unit specifies stress coping candidates that can be carried out, based on the position of the user specified from the position information, and areas where stress coping candidates can be used, and selects a specific stress coping candidate from the specified stress coping candidates based on the stress level of the user.

### Supplementary Note 6

The stress coping management apparatus according to any one of Supplementary Notes 1 to 5,

wherein the stress information obtaining unit further obtains time information related to a time when the user desires stress copings,

the stress coping candidate registration unit registers a stress coping candidate along with information regarding a time when the stress coping candidate can be used, and

the stress coping candidate selecting unit specifies stress coping candidates that can be carried out, based on the time when the user desires stress copings, the time being specified based on the time information, and times when stress coping candidates can be used, and selects a specific stress coping candidate from the specified stress coping candidates based on the stress level of the user.

### Supplementary Note 7

The stress coping management apparatus according to any one of Supplementary Notes 1 to 6,

wherein the stress coping candidate registration unit specifies the number of times a presented stress coping candidate was used by the user, and sets a cost of registration of the stress coping candidate based on the specified number of times.

### Supplementary Note 8

The stress coping management apparatus according to any one of Supplementary Notes 1 to 7,

wherein the stress information obtaining unit obtains the stress information via a server apparatus, and

the stress coping candidate presentation unit presents the selected stress coping candidate to a terminal apparatus.

### Supplementary Note 9

A stress coping management method including:

a stress information obtaining step of obtaining stress information for specifying a stress level of a user;

a stress coping candidate registration step of registering a stress coping candidate in advance;

a stress coping candidate selecting step of selecting a specific stress coping candidate from stress coping candidates registered in advance, based on the stress level of the user specified from the stress information;

a stress coping candidate presentation step of presenting the selected stress coping candidate; and

wherein, in the stress coping candidate registration step, accepting a request to register a new stress coping candidate, and registering the new stress coping candidate input from a requester that has made the request to register the new stress coping candidate, and a stress level corresponding to the new stress coping candidate.

### Supplementary Note 10

The stress coping management method according to Supplementary Note 9,

wherein, in the stress coping candidate presentation step, a weight is set for each stress coping candidate, and

in the stress coping candidate presentation step , when a plurality of stress coping candidates are selected, a priority of each of the plurality of selected stress coping candidates is set in accordance with the weight, and the selected stress coping candidates are presented in priority order.

### Supplementary Note 11

The stress coping management method according to Supplementary Note 9 or 10, further including:

a notification step of notifying of stress levels of the user before and after the stress coping candidate is selected, to a requester that has made a request to register the selected stress coping candidate.

### Supplementary Note 12

The stress coping management method according to any one of Supplementary Notes 9 to 11, further including:

a stress level estimation step of estimating a stress level based on biological information of the user if the stress information includes the biological information,

wherein, in the stress coping candidate selecting step, a specific stress coping candidate is selected based on the stress level estimated from the biological information.

### Supplementary Note 13

The stress coping management method according to any one of Supplementary Notes 9 to 12,

wherein, in the stress information obtaining step, position information for specifying a position of the user is further obtained,

in the stress coping candidate registration step, a stress coping candidate is registered along with information regarding an area where the stress coping candidate can be used, and

in the stress coping candidate selecting step, stress coping candidates that can be carried out are specified based on the position of the user specified from the position information, and areas where stress coping candidates can be used, and a specific stress coping candidate is selected from the specified stress coping candidates based on the stress level of the user.

### Supplementary Note 14

The stress coping management method according to any one of Supplementary Notes 9 to 13,

wherein, in stress information obtaining step, time information related to a time when the user desires stress copings is further obtained,

in the stress coping candidate registration step, a stress coping candidate is registered along with information regarding a time when the stress coping candidate can be used,

in the stress coping candidate selecting step, stress coping candidates that can be carried out are specified based on the time when the user desires stress copings, the time being specified based on the time information, and times when stress coping candidates can be used, and a specific stress coping candidate is selected from the specified stress coping candidates based on the stress level of the user.

### Supplementary Note 15

The stress coping management method according to any one of Supplementary Notes 9 to 14,

wherein, in the stress coping candidate registration step, the number of times a presented stress coping candidate was used by the user is specified, and a cost of registration of the stress coping candidate is set based on the specified number of times.

### Supplementary Note 16

The stress coping management method according to any one of Supplementary Notes 9 to 15,

wherein, in the stress information obtaining step, the stress information is obtained via a server apparatus, and

in the stress coping candidate presentation step, the selected stress coping candidate is presented to a terminal apparatus.

### Supplementary Note 17

A computer-readable recording medium that includes recorded thereon a program,

the program including instructions that cause a computer to carry out:

a stress information obtaining step of obtaining stress information for specifying a stress level of a user;

a stress coping candidate registration step of registering a stress coping candidate in advance;

a stress coping candidate selecting step of selecting a specific stress coping candidate from stress coping candidates registered in advance, based on the stress level of the user specified from the stress information;

a stress coping candidate presentation step of presenting the selected stress coping candidate; and

wherein, in the stress coping candidate registration step, accepting a request to register a new stress coping candidate, and registering the new stress coping candidate input from a requester that has made the request to register the new stress coping candidate, and a stress level corresponding to the new stress coping candidate.

### Supplementary Note 18

The computer-readable recording medium according to Supplementary Note 17,

wherein, in the stress coping candidate presentation step, a weight is set for each stress coping candidate, and

in the stress coping candidate presentation step, when a plurality of stress coping candidates are selected, a priority of each of the plurality of selected stress coping candidates is set in accordance with the weight, and the selected stress coping candidates are presented in priority order.

### Supplementary Note 19

The computer-readable recording medium according to Supplementary Note 17 or 18,

wherein, the program including further instruction that cause the computer to carry out:

a notification step of notifying of stress levels of the user before and after the stress coping candidate is selected, to a requester that has made a request to register the selected stress coping candidate.

### Supplementary Note 20

The computer-readable recording medium according to any one of Supplementary Notes 17 to 19,

wherein the program including further instruction that cause the computer to carry out:

a stress level estimation step of estimating a stress level based on biological information of the user if the stress information includes the biological information,

wherein, in the stress coping candidate selecting step, a specific stress coping candidate is selected based on the stress level estimated from the biological information.

### Supplementary Note 21

The computer-readable recording medium according to any one of Supplementary Notes 17 to 19,

wherein, in the stress information obtaining step, position information for specifying a position of the user is further obtained,

in the stress coping candidate registration step, a stress coping candidate is registered along with information regarding an area where the stress coping candidate can be used, and

in the stress coping candidate selecting step, stress coping candidates that can be carried out are specified based on the position of the user specified from the position information, and areas where stress coping candidates can be used, and a specific stress coping candidate is selected from the specified stress coping candidates based on the stress level of the user.

### Supplementary Note 22

The computer-readable recording medium according to any one of Supplementary Notes 17 to 21,

wherein, in the stress information obtaining step, time information related to a time when the user desires stress copings is further obtained,

in the stress coping candidate registration step, a stress coping candidate is registered along with information regarding a time when the stress coping candidate can be used,

in the stress coping candidate selecting step, stress coping candidates that can be carried out are specified based on the time when the user desires stress copings, the time being specified based on the time information, and times when stress coping candidates can be used, and a specific stress coping candidate is selected from the specified stress coping candidates based on the stress level of the user.

### Supplementary Note 23

The computer-readable recording medium according to any one of Supplementary Notes 17 to 22,

wherein, in the stress coping candidate registration step, the number of times a presented stress coping candidate was used by the user is specified, and a cost of registration of the stress coping candidate is set based on the specified number of times.

### Supplementary Note 24

The computer-readable recording medium according to any one of Supplementary Notes 17 to 23,

wherein, in the stress information obtaining step, the stress information is obtained via a server apparatus, and

in the stress coping candidate presentation step, the selected stress coping candidate is presented to a terminal apparatus.

Although the invention of the present application has been described above with reference to the example embodiment, the invention of the present application is not limited to the above-described example embodiment. Various changes that can be understood by a person skilled in the art within the scope of the invention of the present application can be made to the configuration and the details of the invention of the present application.

### INDUSTRIAL APPLICABILITY

As described above, according to the present invention, it is possible to present stress copings that are not restricted by past records, to a user. The present invention is useful for human stress management system.

### REFERENCE SIGNS LIST

10 Stress coping management apparatus

11 Stress information obtaining unit

12 Stress coping candidate registration unit

13 Stress coping candidate selection unit

14 Stress coping candidate presentation unit

15 Stress level estimation unit

16 Stress coping candidate storage unit

17 Stress coping history storage unit

20 Terminal apparatus of user

21 User

30 Server apparatus

40 Network

50 Terminal apparatus of vendor

51 Vendor

110 Computer

111 CPU

112 Main memory

113 Storage device

114 Input interface

115 Display controller

116 Data reader/writer

117 Communication interface

118 Input device

119 Display device

120 Recording medium

121 Bus

What is claimed is:

1. A stress coping management apparatus comprising:
at least one memory storing instructions; and
at least one processor configured to execute the instructions to:
obtain stress information for specifying a stress level of a user;
register a stress coping candidate in advance;
select a specific stress coping candidate from stress coping candidates registered in advance, based on the stress level of the user specified from the stress information; and
present the selected stress coping candidate,
accept a request to register a new stress coping candidate, and register the new stress coping candidate input from a requester that has made the request to register the new stress coping candidate, and a stress level corresponding to the new stress coping candidate.

2. The stress coping management apparatus according to claim 1,
where, further at least one processor configured to execute the instructions to:
set a weight for each stress coping candidate, and
when a plurality of stress coping candidates are selected, set a priority of each of the plurality of selected stress coping candidates in accordance with the weight, and present the selected stress coping candidates in priority order.

3. The stress coping management apparatus according to claim 1,
wherein, further at least one processor configured to execute the instructions to:
notify a requester that has made a request to register the selected stress coping candidate, of stress levels of the user before and after the stress coping candidate is selected.

4. The stress coping management apparatus according to claim 1,
wherein, further at least one processor configured to execute the instructions to:
estimate a stress level based on biological information of the user if the stress information includes the biological information,
select a specific stress coping candidate based on the stress level estimated from the biological information.

5. The stress coping management apparatus according to claim 1,
wherein, further at least one processor configured to execute the instructions to:
obtain position information for specifying a position of the user,
register a stress coping candidate along with information regarding an area where the stress coping candidate can be used, and
specify stress coping candidates that can be carried out, based on the position of the user specified from the position information, and areas where stress coping candidates can be used, and select a specific stress coping candidate from the specified stress coping candidates based on the stress level of the user.

6. The stress coping management apparatus according to claim 1,
wherein, further at least one processor configured to execute the instructions to:
obtain time information related to a time when the user desires stress copings,
register a stress coping candidate along with information regarding a time when the stress coping candidate can be used, and
specify stress coping candidates that can be carried out, based on the time when the user desires stress copings, the time being specified based on the time information, and times when stress coping candidates can be used, and select a specific stress coping candidate from the specified stress coping candidates based on the stress level of the user.

7. The stress coping management apparatus according to claim 1,
wherein, further at least one processor configured to execute the instructions to:
specify the number of times a presented stress coping candidate was used by the user, and set a cost of registration of the stress coping candidate based on the specified number of times.

8. The stress coping management apparatus according to claim 1,
wherein, further at least one processor configured to execute the instructions to:
obtain the stress information via a server apparatus, and
present the selected stress coping candidate to a terminal apparatus.

9. A stress coping management method comprising:
obtaining stress information for specifying a stress level of a user;
registering a stress coping candidate in advance;
selecting a specific stress coping candidate from stress coping candidates registered in advance, based on the stress level of the user specified from the stress information;
presenting the selected stress coping candidate; and
accepting a request to register a new stress coping candidate, and registering the new stress coping candidate input from a requester that has made the request to register the new stress coping candidate, and a stress level corresponding to the new stress coping candidate.

10-16. (canceled)

17. A non-transitory computer-readable recording medium that includes a program recorded thereon, the program including instructions that cause a computer to:
obtain stress information for specifying a stress level of a user;
register a stress coping candidate in advance;
select a specific stress coping candidate from stress coping candidates registered in advance, based on the stress level of the user specified from the stress information;
present the selected stress coping candidate; and
accept a request to register a new stress coping candidate, and register the new stress coping candidate input from a requester that has made the request to register the new stress coping candidate, and a stress level corresponding to the new stress coping candidate.

18-24. (canceled)
